(19) United States
(12) Patent Application Publication
(10) Pub. No.: US 2020/0246414 A1
(43) Pub. Date: Aug. 6, 2020

(54) COMPOSITIONS AND METHODS FOR THE TREATMENT OF CONSTIPATION AND OTHER AILMENTS OF THE GASTROINTESTINAL SYSTEM

(71) Applicant: K.L.R.M., LLC, San Pedro, CA (US)

(72) Inventor: Jacob Rajfer, San Pedro, CA (US)

(21) Appl. No.: 16/782,965

(22) Filed: Feb. 5, 2020

Related U.S. Application Data
(60) Provisional application No. 62/801,828, filed on Feb. 6, 2019.

(51) Int. Cl. A61K 36/906; A61K 36/77; A61K 9/00; A61K 9/20; A61K 9/48; A61K 31/198; A61K 31/17; A61P 1/10

(52) U.S. Cl. CPC A61K 36/906; A61K 36/77; A61K 9/0056; A61P 1/10; A61K 9/4808; A61K 31/198; A61K 31/17; A61K 9/20

(57) ABSTRACT

A method for treatment of constipation and other ailments of the gastrointestinal system includes administering a pharmaceutically effective amount of a composition over a sufficient period of time that results in the production of nitric oxide in gastrointestinal cells and ultimately has beneficial results in treating constipation and other ailments of the gastrointestinal system. The composition comprises ginger or a ginger derivative, muira puama, and paullinia cupana that stimulates nitric oxide in gastrointestinal cells to increase NO and improve bowel functioning thereof.

### CROSS REFERENCE TO RELATED APPLICATION

This application claims the benefit of U.S. Provisional Patent Application Ser. No. 62/801,828, entitled “COMPOSITIONS AND METHODS FOR THE TREATMENT OF CONSTIPATION AND OTHER AILMENTS OF THE GASTROINTESTINAL SYSTEM,” filed Feb. 6, 2019.

### BACKGROUND OF THE INVENTION

### 1. Field of the Invention

The present invention relates to compositions and methods for the treatment of constipation and other ailments of the gastrointestinal system.

### 2. Description of the Related Art

Constipation affects about 14% of people worldwide. In the United States, about 15% of adults and 30% of people older than 60 years suffer from constipation which is defined as having hard, dry bowel movements, or going fewer than three times a week. The actual prevalence is likely larger as many people believe their bowel habits are normal and underreport symptoms of constipation. Moreover, most recent surveys assessing current medical management of constipation show that patients are dissatisfied and believe that conventional treatments, like laxatives and colonic stimulants, are suboptimal since they can cause explosive bowel movements which are difficult to control. The average patient with constipation tries approximately 4 over-the-counter and 2 prescription medications before finding an effective treatment. In spite of this, most patients report that the relief they receive is unacceptable.

In addition, individuals affected by constipation are reported to have significant rates of absenteeism at work. Those suffering with constipation also have poorer general health, mental health, and social functioning when compared with healthy controls. They also report that the constipation interferes with enjoyable activities (eg, family functions, children's school or sporting events) and has negatively affected their self-confidence, ability to engage in hobbies they enjoyed in the past, partnership relationships and intimacy, and job/career or ability to work.

Irritable bowel syndrome (IBS) is a functional gastrointestinal disorder characterized by altered bowel habits and abdominal pain that adversely affect quality of life. IBS is sub-classified as constipation-predominant (IBS-C), diarrhea-predominant (IBS-D), or mixed symptom (IBS-M). There is no cure for IBS, and current treatment strategies often require patients to take multiple medications to control their symptoms. Bulking agents, laxatives, and antidiarrheals are prescribed to help normalize alterations in bowel habits, while tricyclic antidepressants and antispasmodics aim to minimize visceral pain associated with the disease. The variable effectiveness of this symptom-targeted approach to IBS treatment underscores the need for alternatives.

### SUMMARY OF THE INVENTION

According to a first aspect there may be provided a method for treatment of constipation and other ailments of the gastrointestinal system. The method includes administering a pharmaceutically effective amount of a composition over a sufficient period of time that results in the production of nitric oxide in gastrointestinal cells and ultimately has beneficial results in treating constipation and other ailments of the gastrointestinal system. The composition comprises ginger or a ginger derivative, muira puama, and paullinia cupana that stimulates nitric oxide in gastrointestinal cells to increases NO and improve functioning thereof.

In some embodiments the composition comprises 10 mg to 3 g ginger or ginger derivative.

In some embodiments the composition further includes L-arginine, L-citrulline, or a mixture of L-arginine and L-citrulline.

In some embodiments the composition comprises 10 mg to 3 g of L-arginine, L-citrulline, or a mixture of L-arginine and L-citrulline.

In some embodiments the composition comprises 62.5 mg to 3 g of muira puama.

In some embodiments the composition comprises 500 mg to 1.5 g of muira puama.

In some embodiments the composition comprises 62.5 mg to 3 g of paullinia cupana.

In some embodiments the composition comprises 500 mg of paullinia cupana.

In some embodiments the composition comprises 10 mg to 3 g of ginger or ginger derivative, 10 mg to 3 g of L-arginine, L-citrulline, or a mixture of L-arginine and L-citrulline, 62.5 mg to 3 g of muira puama, and 62.5 mg to 3 g of paullinia cupana.

In some embodiments the composition comprises 500 mg of ginger or ginger derivative, 1,500 mg or 1,600 mg of L-arginine, L-citrulline, or a mixture of L-arginine and L-citrulline, 500 mg of muira puama, and 500 mg of paullinia cupana.

In a further aspect there may be provided a pharmaceutical composition consisting of an effective amount of ginger or ginger derivative, an effective amount of L-citrulline, L-arginine, or a mixture of L-arginine and L-citrulline, an effective amount of muira puama, and an effective amount of paullinia cupana. The composition is administered in periodic dosages for a sufficient period of time to treat constipation and other ailments of the gastrointestinal system, and the composition is in an oral dosage form comprising a tablet, capsule, or lozenge.

In another aspect there may be provided a pharmaceutical composition consisting of an effective amount of ginger or ginger derivative, an effective amount of muira puama, and an effective amount of paullinia cupana. The composition is administered in periodic dosages for a sufficient period of time to treat constipation and other ailments of the gastrointestinal system, and the composition is in an oral dosage form comprising a tablet, capsule, or lozenge.

Additional advantages of the embodiments will be set forth in part in the description which follows, and in part will be understood from the description, or may be learned by practice of the invention. The advantages will be realized and attained by means of the elements and combinations particularly pointed out in the appended claims.

### BRIEF DESCRIPTION OF THE DRAWINGS

FIG. 1 shows test data regarding Bristol stool score (p<0.01) and stools per week (p<0.01) pre- and post-treatment with the nutraceutical composition of the present invention.

FIG. 2 shows test data regarding the sensation of stool evacuation and abdominal pain pre- and post-treatment with the nutraceutical composition of the present invention.

FIG. 3 shows test data regarding bowel movements before and after taking the nutraceutical composition of the present invention.

FIG. 4 shows test data regarding an increase in average weekly bowel movements before and after taking the nutraceutical composition of the present invention for 2-4 weeks from 2.7 to 7.3 with p<0.0001.

FIG. 5 shows test data regarding improvements in the sensation of bowel evacuation before and after taking the nutraceutical composition of the present invention.

FIG. 6 shows test data regarding Bristol stool score (p<0.001) before and after treatment with the nutraceutical composition of the present invention increased from 1.5 to 3.4.

FIG. 7 shows test data regarding a decrease in the number of patients with abdominal pain before and after the present invention.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as a basis for teaching one skilled in the art how to make and/or use the invention.

The present invention provides compositions and methods for the treatment of constipation and other ailments of the gastrointestinal system. In accordance with a first embodiment, the present invention provides a nutraceutical composition for treating constipation and other ailments of the gastrointestinal system (which is referenced herein and in various figures as COMB-4) that comprises effective amounts of ginger or a ginger derivative selected from the group consisting of fresh, partially dried vegetable ginger, dried vegetable ginger, 6-gingerol and mixtures thereof; at least one of the group consisting of arginine and citrulline (preferably, L-arginine, L-citrulline, or a combination of L-arginine and L-citrulline); muira puama; and paullinia cupana (guarana).

It is known that nitric oxide (NO) is the chemical that is important for the normal functioning of the mucosa (secretes the fluid to the stool) as well as the vascular tone of the gastrointestinal tissue. Further, NO has recently been shown to be a neurotransmitter in the nonadrenergic, noncholinergic (NANC) nerves of the human gut. As a result, NO from these NANC nerves participates in the modulation of the smooth musculature tone, such as the regulation of intestinal peristalsis, gastric emptying and antral motor activity. NO also regulates acid and gastric mucus secretion, alkaline production and is involved in the maintenance of mucosal blood flow. Nitin I. Kochar, Anil V. Chandewal, Ravindra L. Bakal and Priya N. Kochar, 2011. Nitric Oxide and the Gastrointestinal Tract. International Journal of Pharmacology, 7: 31-39.

As a signaling molecule throughout the body, NO activates soluble guanylate cyclase to form cGMP, and it is cGMP that is the 2^{nd} messenger in the NO-cGMP pathway that is responsible for all the effects of NO in the GI tract. Brasitus, T. A., M. Field and D. V. Kimberg, 1976. Intestinal mucosal cyclic GMP: Regulation and relation to ion transport. Am. J. Physiol., 231: 275-282. Nitric oxide donors, such as sodium nitroprus side, S-nitroso-N-acetylpenicillamine and isosorbide dinitrate, stimulated mucus secretion from a suspension of isolated gastric cells. Brown, J. F., B. L. Tepperman, P. J. Hanson, B. J. Whittle and S. Moncada, 1992. Differential distribution of nitric oxide synthase between cell fractions isolated from the rat gastric mucosa. Biochem. Biophy. Res. Commun., 184: 680-685. Dibutyryl cyclic GMP and the cyclic GMP phosphodiesterase inhibitor M and B 22948 also increased the mucus release. Topical administration of the NOS inhibitor N-monomethyl-L-arginine reduced fluid secretion in patients with collagenous colitis. Perner, A., L. Andresen, M. Normark, B. Fischer-Hansen, J. Rask-Madsen, J. Eugen-Olsend and J. Rask-Madsena, 2001. Expression of nitric oxide synthases and effects of L-arginine and L-NMMA on nitric oxide production and fluid transport in collagenous colitis. Gut, 49: 387-394.

As Applicant's prior work shows, COMB-4 is known to stimulate the NOS enzymes which make NO in various other cells; for example, COMB-4 stimulates iNOS in the cavernosal smooth muscle cell (see U.S. Patent Application Publication No. 2014/0255528, entitled “COMPOSITIONS AND METHODS FOR TREATING, INHIBITING THE ONSET, AND SLOWING THE PROGRESSION OF ERECTILE DYSFUNCTION INCLUDING NATURALLY OCCURRING AGE RELATED ERECTILE DYSFUNCTION”, which is incorporated herein by reference) and COMB-4 stimulates both iNOS and eNOS in the bone (see U.S. Patent Application Publication No. 2018/0104300, entitled “COMPOSITIONS AND METHODS FOR THE TREATMENT OF ORTHOPEDIC AILMENTS”, which is incorporated herein by reference). Applicant's in vitro testing, which is presented below, demonstrates that COMB-4 is effective in encouraging the production of NO in gastrointestinal cells and ultimately has beneficial results in treating constipation and other ailments of the gastrointestinal system.

Evidence generated from experiments and epidemiologic studies in humans suggest that exogenous NO has a protective effect on the gastrointestinal tract. The inflammatory response that occurs within the GI tract with inflammatory bowel disease (IBD) is associated with induction of iNOS and increased NO production in the gut. Although the precise effect of iNOS induction in these experimental settings remains unclear, it has been shown that NO at least is required to heal and protect intestinal lesions from some of these inflammatory conditions. The major clinical hallmarks of IBS are alterations in intestinal motility, secretion, and visceral sensation. Although the underlying cause of IBS is unknown, it is well-established that cyclic guanosine monophosphate (cGMP), which is formed from the activation of its enzyme guanylyl cyclase by NO, activates secretion of fluids in the intestine. Indeed, when PDE5 inhibitors which upregulate the effect of the NO-cGMP pathway are used in IBS, improvement in intestinal motility and secretion occurs.

Considering the individual components of the nutraceutical composition in accordance with the present invention, ginger is a complex natural composition having numerous purported properties when used alone and/or in combination with other compounds. For example, some traditional Chinese medicines have used or included ginger in compositions to treat or prevent various maladies based on a variety of metaphysical reasons. However, over the past century, scientific methods have shown that many traditional Chinese medicines do not produce the purported effects and/or may even make the target maladies worse. Nevertheless, some traditional Chinese medicines have been found to contain active agents that may be of medical use, even if not effective or safe for the use purported by traditional Chinese medicine. The complexity of ginger and its myriad properties is reflected by certain constituent compounds which have the following structure:

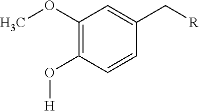

wherein, for example, in 6-gingerol the R sidechain of the vanillyl function group (i.e., 4-hydroxy-3-methoxyphenyl group) is:

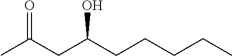

Thus, 6-gingerol (also called gingerol) is (S)-5-hydroxy-1-(4-hydroxy-3-methoxyphenyl)-3-decanone and has the following structure:

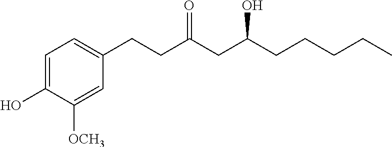 [SMILES: CCCCC[C@H](O)CC(=O)CCc1ccc(O)c(OC)c1]

Since ginger contains multiple compounds, of varying complexity and chemical activity, there are conflicting teachings in the art about the biological activity of compounds that might be useful in inducing nitric oxide production or otherwise having a potential role in treatment. Further, there remains considerable unpredictability about how to understand, much less control, the relevant metabolic pathways.

As previously mentioned, Applicant has discovered that ginger, when combined with muira puama, paullinia cupana (guarana), and at least one of the group consisting of arginine and citrulline (preferably, L-arginine, L-citrulline, or a combination of L-arginine and L-citrulline) is effective in treating constipation and other ailments of the gastrointestinal system.

An oral dosage form of the nutraceutical composition in accordance with the present invention is selected from the group consisting of a tablet, capsule, lozenge, powder or suspension comprising the foregoing ingredients. The raw materials and ingredient matter may be dried, for example by freeze-drying or vacuum drying, before compounding into oral dosage forms. Individual dosage forms may comprise compressed tablets, capsules, lozenges or may be provided in sachets. Suspension formulations may be provided. Ginger, ginger root extract, L-arginine, L-citrulline, muira puama and paullinia cupana (guarana) are all separately commercially available. Preferably, the ingredients are combined and encapsulated in gelatin capsules, but other dosage forms are anticipated that will produce equivalent results.

Flavorings or taste masking agents may be employed. Tablets or other dosage forms may include diluents (e.g., lactose), disintegrants (e.g., cross carmelose sodium), or binders (e.g., polyvinylpyrollidone). Lubricants for example magnesium stearate, or other conventional excipients may be employed (e.g., silicas, carbohydrates, etc.). Film-coated tablets may be provided.

The active ingredients of compositions of the present invention are combined using well known and standard processes and agents. Preferably, a gelatin capsule contains the combined ingredients in powder form. Standard ingredients in powder formulations are used for preparing and compounding preferred exemplary formulations of the present invention. The ingredients, in powder form, are inspected, weighed, blended and encapsulated in gelatin capsules. The blending process includes standard screening, blending and metal detection at standard temperatures and in a sterile environment at least sufficient for food supplements.

Since certain preferred formulations of the present nutraceutical composition comprise compounds found in foods or extracted from foods, they may be referred to as “nutraceuticals.” While nutraceutical compositions have traditionally been found in a medicinal format, such as capsules or tablets, an increasing number of foods have been fortified with nutraceuticals. Analogs and/or homologs of ginger constituents in combination with muira puama, paullinia cupana (guarana), and L-arginine and/or L-citrulline are used in accordance with the present invention to promote nitric oxide production within the gastrointestinal system sufficient to treat constipation and other ailments of the gastrointestinal system. Though a gelatin capsule with the constituents of the nutraceutical composition is preferred for administering, the present nutraceutical composition can be administered in a wide variety of ways and forms matching the lifestyle and dietary preferences of the users, as once or twice a day dietary supplements, mixed into foods or “smoothies,” etc.

“Effective amount” as used in the present disclosure is intended to mean that a dosage form of the nutraceutical composition contains an amount of each ingredient sufficient when administered to a human patient for a sufficient period of time to treat constipation. The “beneficial effect” includes, in the context of treating constipation, at least one of improved sensation of complete evacuation, improved Bristol stool score, decreased straining during defecations, and increased bowel movement frequency.

“Individual Dosage” as used herein means the amount of the nutraceutical composition in a single dose of the nutraceutical composition administered to a human patient as part of a dosing regimen.

“Total Daily Dosage” as used herein means the cumulative amount of the nutraceutical composition administered to a human patient over the course of a day whether the nutraceutical composition is administered once a day or multiple times a day as part of a dosing regimen.

“About” as used herein refers to a range of values +/−10% of a specified value.

In accordance with a preferred embodiment, the total daily dosage of the nutraceutical composition is as follows:
about 10 mg to about 3 g ginger or ginger derivative,
about 187.5 mg to about 3 g of a L-citrulline, L-arginine or a combination of L-arginine and L-citrulline,
about 62.5 mg to about 3 g (preferably about 500 mg to about 1.5 g) muira puama, and
about 62.5 mg to about 3 g paullinia cupana (guarana).

A specific individual dosage forming the basis for the test results presented herein, where the nutraceutical composition is taken twice a day, includes
about 250 mg ginger or ginger derivative,
about 750 mg (or 800 mg as dictated by manufacturing constraints) L-citrulline,
about 250 mg muira puama, and
about 250 mg paullinia cupana (guarana).

As such, this results in a preferred daily dosage of
about 500 mg ginger or ginger derivative,
about 1,500 mg (or 1,600 mg as dictated by manufacturing constraints) L-citrulline,
about 500 mg muira puama, and
about 500 mg paullinia cupana (guarana).

While a twice daily administration of the nutraceutical composition of the present invention is preferred, it is appreciated the nutraceutical composition may be administered using other dosing regimens and the individual dosages would therefore be adjusted so as to not exceed the preferred total daily dosage as outlined above. Whether the nutraceutical composition is administered once a day or multiple times throughout the day, the nutraceutical composition is administered for a sufficient period of time to treat constipation.

In accordance with an alternate embodiment of the present invention, constipation and other ailments of the gastrointestinal system may also be treated with a variation of COMB-4 wherein the L-citrulline, L-arginine or combination of L-arginine and L-citrulline is removed, referred to herein as COMB-3. Preliminary tests reveal that COMB-3, while not producing beneficial results to the extent found in accordance with COMB-4, does assist in treating constipation and other ailments of the gastrointestinal system. The removal of L-citrulline, L-arginine or combination of L-arginine and L-citrulline from the composition results in a reduction in the size of the pills and/or capsules a patient would require, which is believed to potentially make it more likely a patient would take the nutraceutical composition as required for achieving noticeable improvements with regards to constipation and other ailments of the gastrointestinal system.

In accordance with such an embodiment, the nutraceutical composition comprises effective amounts of ginger or a ginger derivative selected from the group consisting of fresh, partially dried vegetable ginger, dried vegetable ginger, 6-gingerol and mixtures thereof; muira puama; and paullinia cupana (guarana).

In accordance with a preferred embodiment of this alternate nutraceutical composition, the total daily dosage of the nutraceutical composition is:
about 10 mg to about 3 g ginger or ginger derivative,
about 62.5 mg to about 3 g (preferably about 500 mg to about 1.5 g) muira puama, and
about 62.5 mg to about 3 g paullinia cupana (guarana).

A specific individual dosage of COMB-3 forming the basis for the test results presented herein, where the nutraceutical composition is taken only once a day, includes:
about 250 mg ginger or ginger derivative,
about 250 mg muira puama, and
about 250 mg paullinia cupana (guarana).

As such, this results in a preferred daily dosage of
about 500 mg ginger or ginger derivative,
about 500 mg muira puama, and
about 500 mg paullinia cupana (guarana).

The nutraceutical composition may include optional pharmaceutically acceptable excipients, fillers, binders, and colorants, and can be packaged in standard gelatin capsules or formed into solid tablets, taken in particulate form, or mixed into and/or suspended in solution.

The present nutraceutical composition reflects the ability of a combination of ginger, muira puama, paullinia cupana (guarana), and L-citrulline and/or L-arginine (or the three constituent nutraceutical composition as described above) to treat constipation. The nutraceutical composition may be taken for an indefinite period to sustain the beneficial effects.

Efficacy of the present nutraceutical composition in the treatment of constipation and other ailments of the gastrointestinal system was tested as follows.

### Methods

Ten patients with severe constipation were treated with COMB-4 (that is, the present composition at a daily dosage of 500 mg/day ginger rhizome, 1,500 mg/day of L-Citrulline, 500 mg/day muira puama, and 500 mg/day paullinia cupana (guarana) in a 70 Kg man) for 1-4 weeks. Prior to initiation of COMB-4 therapy, all patients were on multiple laxatives and experienced at least two or more of the following without laxatives: sensation of incomplete evacuation, Bristol stool score 1-2 and straining during the majority of defecations. Data were collected with regards to pre- and post-treatment symptoms, including: Bristol stool score, bowel movement frequency, and sensation of evacuation. Wilcoxon rank test was used for statistical analysis.

### Results

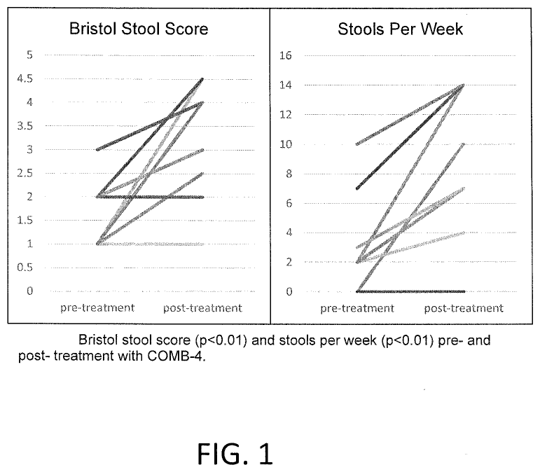

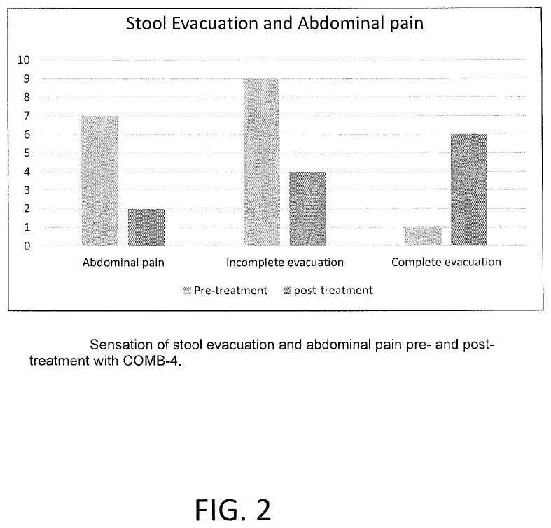

The mean Bristol stool score increased from 1.5 to 3.3 (p<0.01, FIG. 1). The mean number of stools per week increased from 3.3 to 8.4 (p<0.01, FIG. 1) post-treatment. Eight out of ten patients were free of abdominal pain (FIG. 2) and 6/10 patients felt the sensation of complete stool evacuation with bowel movements post-treatment (FIG. 2). Seven out of ten patients stopped all other laxatives after initiation of COMB-4 therapy.

### Conclusion

The mixture of ginger, L-citrulline, muira puama, and paullinia cupana (guarana) (COMB-4) could be a useful addition to the management options for severe constipation.

Further testing regarding the efficacy of the present nutraceutical composition in the treatment of constipation and other ailments of the gastrointestinal system was conducted as follows.

### Methods

Seventeen patients with constipation were treated with COMB-4 (2-4 capsules per day, wherein each capsule is composed of 125 mg ginger, 400 mg L-citrulline, 125 mg muira puama, and 125 mg paullinia cupana (guarana)) for 2-4 weeks. Constipation was defined as two or more of the following: the sensation of incomplete evacuation, Bristol stool score 1-2 (hard, lumpy nut or sausage-shaped BM), and straining during the majority of defecations. Prichard, D. O., & Bharucha, A. E. (2018). Recent advances in understanding and managing chronic constipation. F1000Research, 7, 1640. doi:10.12688/f1000research.15900.1. Patients were surveyed before and after the treatment about their approximate number of bowel movements per week, their typical Bristol stool score, the presence of abdominal pain, and the feeling of incomplete evacuation. The Wilcoxon rank test was used for statistical analysis.

### Results

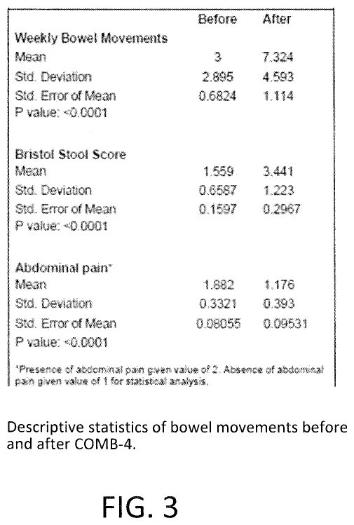

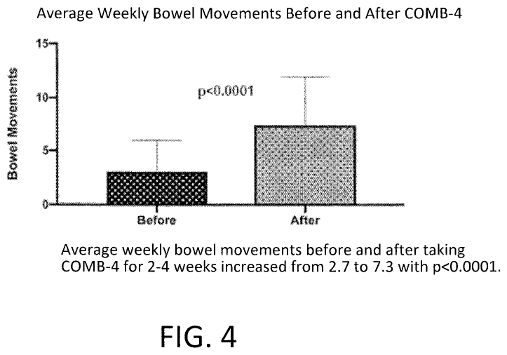

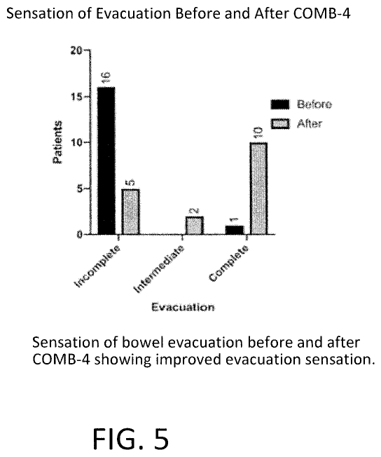

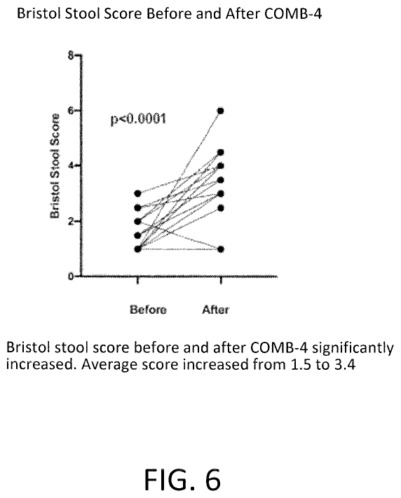

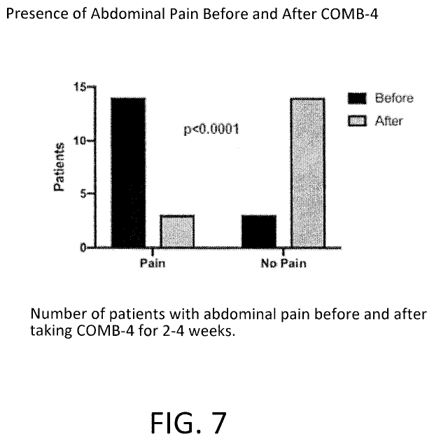

Following treatment with COMB-4, average weekly bowel movements, Bristol stool score, and abdominal pain all improved significantly with p<0.0001 (FIG. 3). The mean number of weekly stools increased from 2.7 to 7.3 (FIG. 4). Patients reported an improved sensation of complete stool evacuation after COMB-4 treatment (FIG. 5). The mean Bristol Stool Score increased from 1.5 to 3.4 with softer and more formed stools (FIG. 6) and patients experienced a significant decrease in abdominal pain (FIG. 7).

### Conclusion

The combination of ginger, paulinia cupana, muira puama, and L-citrulline improves the frequency and consistency of stools and help relieve symptoms of constipation. The nutraceutical may be a novel agent used to treat severe constipation not responding well to other conventional treatments.

While details of certain embodiments of the present inventions are described, they are provided as illustrative examples so as to enable those of ordinary skill in the art to practice the inventions. The details provided are not meant to limit the scope of the present inventions, but to be exemplary. Where certain elements of the present inventions can be partially or fully implemented using known constituents, only those portions of such known constituents that are necessary for an understanding and making of the present invention are described, and detailed descriptions of other constituents or formulating processes are omitted to simplify explanation of the invention. Further, the present invention encompasses present and future known equivalents to the compositions and methods referred to herein. The inventions are capable of other embodiments and of being practiced and carried out in various ways, and as such, those skilled in the art will appreciate that the conception upon which this disclosure is based may readily be utilized as a basis for the designing of other methods and compositions for carrying out the several purposes of the present inventions.

1. A method for treatment of constipation and other ailments of the gastrointestinal system, comprising:
administering a pharmaceutically effective amount of a composition over a sufficient period of time that results in the production of nitric oxide in gastrointestinal cells and ultimately has beneficial results in treating constipation and other ailments of the gastrointestinal system, wherein the composition comprises ginger or a ginger derivative, Muira puama, and Paullinia cupana that stimulates nitric oxide in gastrointestinal cells to increases NO and improve functioning thereof.

2. The method according to claim 1, wherein the composition comprises 10 mg to 3 g ginger or ginger derivative.

3. The method according to claim 1, wherein the composition further includes L-arginine, L-citrulline, or a mixture of L-arginine and L-citrulline.

4. The method according to claim 3, wherein the composition comprises 10 mg to 3 g of L-arginine, L-citrulline, or a mixture of L-arginine and L-citrulline.

5. The method according to claim 4, wherein the composition comprises 10 mg to about 3 g of ginger or ginger derivative, 62.5 mg to about 3 g of muira puama, and 62.5 mg to 3 g of paullinia cupana.

6. The method according to claim 1, wherein the composition comprises 62.5 mg to 3 g of muira puama.

7. The method according to claim 6, wherein the composition comprises 500 mg to 1.5 g of muira puama.

8. The method according to claim 1, wherein the composition comprises 62.5 mg to 3 g of paullinia cupana.

9. The method according to claim 1, wherein the composition comprises 500 mg of paullinia cupana.

10. The method according to claim 1, wherein the composition comprises 10 mg to 3 g of ginger or ginger derivative, 62.5 mg to 3 g of muira puama, and 62.5 mg to 3 g of paullinia cupana.

11. The method according to claim 1, wherein the composition comprises 500 mg of ginger or ginger derivative, 500 mg of muira puama, and 500 mg of paullinia cupana.

12. The method according to claim 1, wherein the composition consists of ginger or ginger derivative, muira puama, and paullinia cupana.

13. The method according to claim 12, wherein the composition consists of 10 mg to 3 g of ginger or ginger derivative, 62.5 mg to 3 g of muira puama, and 62.5 mg to 3 g of paullinia cupana.

14. The method according to claim 13, wherein the composition consists of 500 mg of ginger or ginger derivative, 500 mg of muira puama, and 500 mg of paullinia cupana.

15. A pharmaceutical composition, consisting of:
an effective amount of ginger or ginger derivative,
an effective amount of L-citrulline, L-arginine, or a mixture of L-arginine and L-citrulline,
an effective amount of muira puama, and
an effective amount of paullinia cupana,
wherein the composition can be administered in periodic dosages for a sufficient period of time to treat constipation and other ailments of the gastrointestinal system, and the composition is in an oral dosage form comprising a tablet, capsule, or lozenge.

16. The pharmaceutical composition according to claim 15, wherein the composition consists of 10 mg to 3 g of L-arginine, L-citrulline, or a mixture of L-arginine and L-citrulline; 10 mg to about 3 g of ginger or ginger derivative, 62.5 mg to about 3 g of muira puama, and 62.5 mg to 3 g of paullinia cupana.

17. The pharmaceutical composition according to claim 16, wherein the composition consists of 1,500 mg or 1,600 mg of L-arginine, L-citrulline, or a mixture of L-arginine and L-citrulline; 500 mg of ginger or ginger derivative, 500 mg of muira puama, and 500 mg of paullinia cupana.

18. A pharmaceutical composition, consisting of:
an effective amount of ginger or ginger derivative,
an effective amount of muira puama, and
an effective amount of paullinia cupana,
wherein the composition can be administered in periodic dosages for a sufficient period of time to treat constipation and other ailments of the gastrointestinal system, and the composition is in an oral dosage form comprising a tablet, capsule, or lozenge.

19. The pharmaceutical composition according to claim 18, wherein the composition consists of 10 mg to about 3 g of ginger or ginger derivative, 62.5 mg to about 3 g of muira puama, and 62.5 mg to 3 g of paullinia cupana.

20. The pharmaceutical composition according to claim 19, wherein the composition consists of 500 mg of ginger or ginger derivative, 500 mg of muira puama, and 500 mg of paullinia cupana.
